(19) United States
(12) Patent Application Publication
(10) Pub. No.: US 2024/0043370 A1
(43) Pub. Date: Feb. 8, 2024

(54) METHOD FOR TRIETHYLENETETRAMINE PURIFICATION

(71) Applicant: PhilERA New Zealand Ltd., Auckland (NZ)

(72) Inventor: Joseph FORTUNAK, Silver Spring, MD (US)

(21) Appl. No.: 18/230,978

(22) Filed: Aug. 7, 2023

Related U.S. Application Data
(60) Provisional application No. 63/395,829, filed on Aug. 7, 2022.

(51) Int. Cl. C07C 209/84

(52) U.S. Cl. CPC C07C 209/84

(57) ABSTRACT

Methods for the purification of impure triethylenetetramine into highly pure triethylenetetramine are described. In particular, methods for the preparation of relatively pure salts of triethylenetetramine starting from crude triethylenetetramine and crude salts of trethylenetetramine are described.

### RELATED APPLICATIONS

This application claims the benefit of U.S. Provisional No. 63/395,829, filed Aug. 7, 2022, the contents of which are herein incorporated by reference.

### FIELD OF THE INVENTION

Disclosed herein are methods for the purification of crude triethylenetetramine into highly pure triethylenetetramine. Specifically, the invention relates to a method for the preparation of salts of triethylenetetramine starting from crude triethylenetetramine.

### BACKGROUND

Trientine, chemically known as triethylenetetramine or N,N′-bis(2-aminoethyl)-1,2-ethanediamine belongs to the class of polyethylene polyamines. Trientine dihydrochloride is a chelating agent which is used to bind and remove copper in the body in the treatment of Wilson's disease. Trientine dihydrochloride formulation, developed by Aton with the proprietary name SYPRINE, was approved by US FDA on Nov. 8, 1985 for the treatment of patients with Wilson's disease, who are intolerant to penicillamine. Trientine dihydrochloride, due to its activity on copper homeostasis, is being studied for various potential applications in the treatment of various diseases involving copper-mediated pathways. The disuccinate salt of triethylenetetramine (triethylenetetramine succinate; or TES) possesses excellent properties, including: i) a high degree of crystallinity in the solid state; ii) only a single known polymorph; iii) very high purity; iv) excellent ability to reject related impurities during the process of crystallization and v) long-term stability. U.S. Pat. No. 7,582,796 refers to the synthesis of the disuccinate salt of triethylenetrtramine.

Highly complex synthetic methods for the preparation of triethylenetetramine (TET) and the corresponding dihydrochloride salt as well as other salts have been described. U.S. Patent No. U.S. Pat. No. 4,806,517 discloses the synthesis of triethylenetetramine from ethylenediamine and monoethanolamine using Titania supported phosphorous catalyst while U.S. Pat. Nos. 4,550,209 and 5,225,599 disclose catalytic condensation of ethylenediamine and ethylene glycol for the synthesis of linear triethylenetetramine using catalysts like zirconium trimethylene diphosphonate, or metatungstate composites of titanium dioxide and zirconium dioxide. U.S. Pat. No. 4,503,253 discloses the preparation of triethylenetetramine by reaction of an alkanolamine compound with ammonia and an alkyleneamine having two primary amino groups in the presence of a catalyst, such as supported phosphoric acid wherein the support is comprised of silica, alumina or carbon. U.S. Pat. No. 7,960,591 describes a process to make triethyleneaminetetramine by hydrogenation of ethylenediaminediacetonitrile (EDDN) over a catalyst. Each of the aforementioned methods for the preparation of triethylenetetramine, however, requires high temperatures and pressure. Further, due to the various possible side reactions and consequent associated impurities, for each of the aforementioned methods it is difficult to control the purity of the desired amine. Chinese patent CN 102924289 discloses a process for trientine dihydrochloride comprising reduction of N,N′-dibenzyl-,N,N′-bis[2-(1,3-dioxo-2H-isoindolyl)ethyl]ethanediamine using hydrazine hydrate to form N,N′-dibenzyl-,N,N′-bis(2-aminoethyl)ethanediamine, which, upon condensation with benzyl chloroformate gave N,N′-dibenzyl-,N,N′-bis[2-(Cbz-amino)ethyl]ethanediamine, and further reductive deprotection to form the desired compound.

Czechoslovakia patent CS 197,093 refers to a process comprising reaction of triethylenetetramine with concentrated hydrochloric acid to obtain the crystalline tetrahydrochloride salt. Further reaction of the salt with sodium ethoxide in solvent ethanol, filtration of the solid sodium chloride which is generated in the process, followed by slow cooling and crystallization of the filtrate provided the dihydrochloride salt. Optionally, aqueous solution of the tetrahydrochloride salt was passed through a column of an anion exchanger and the eluate containing free base was treated with a calculated amount of hydrochloric acid, evaporated, and the residue was crystallized from aqueous ethanol to yield the dihydrochloride salt. The aforementioned process, however, is not commercially practicable because it is very long and cumbersome, requires the use of strong bases, involves filtration of sodium chloride, and results in low yields (e.g. 60%).

U.S. Pat. No. 8,394,992 refers to a method for preparation of triethylenetetramine dihydrochloride wherein tertiary butoxycarbonyl (boc) protected triethylenetetramine is first converted to its tetrahydrochloride salt using large excess of hydrochloric acid in solvent isopropanol, followed by treatment of the resulting tetrahydrochloride salt with a strong base like sodium alkoxide to produce the amine free base (TETA) and sodium chloride under anhydrous conditions. The free amine is extracted with tertiary butyl methyl ether (TBME), followed by removal of sodium chloride and finally the amine free base TET is treated with hydrochloric acid in solvent ethanol to form trientine hydrochloride salt. The aforementioned method, however, suffers from the following drawbacks; a) Lengthy process comprising treatment of the tetrahydrochloride salt with a base in anhydrous conditions to obtain the amine and its further conversion to TETA dihydrochloride, which includes a number of unit operations such as solvent extraction, washing of filtered solid, solvent concentration, crystallization at various stages of synthesis etc. b) Use of excessive amounts of hydrochloric acid as well as anhydrous alcoholic and ether solvents. c) Stringent requirement of complete removal of sodium chloride formed during the process. If the salt is not scrupulously removed, the final product, trientine hydrochloride salt is unlikely to pass the sulphated ash test, which is indicative of complete removal of inorganic impurities from the drug product.

Korean Patent KR 20080022940 refers to a method for the preparation of triethylenetetramine dihydrochloride salt by dissolving triethylenetetramine free base in methanol and the dihydrochloride salt is formed by addition of aqueous concentrated hydrochloric acid. After methanol removal, the mixture is precipitated out with isopropanol and then filtered to yield a mixture of triethylenetetramine dihydrochloride and an impurity. The impure hydrochloride salt was then recrystallized.

Some reports (Journal of Chemical Research 2005 (4), 233-235; XP055551215)) refer to a method for preparation of trientine dihydrochloride from technical grade triethylenetetramine free base and identifies three impurities [N,N-bis-(2-aminoethyl)-1,2-ethanediamine, 1,4-piperazine diethanamine and N-[2-(1-piperazinyl)ethyl]-1,2-ethanediamine usually formed during its synthesis. The brief method lacks sufficient details to fully obtain the isolated material. Further, there is no mention about the conditions in which the desired product or its impurities are likely to be formed. Also, the dihydrochloride salt obtained is again purified by recrystallization from 95% ethanol wherein insoluble trihydrochloride salt is removed as insoluble impurity during the recrystallization process.

European patent EP 3 350 154 B1 describes an expensive process for the synthesis of triethylenetetramine dihydrochloride comprising a single step of treating Boc-protected amine with hydrochloric acid to form the desired dihydrochloride salt in substantially pure form and in good yield.

All of the aforementioned references are incorporated by reference in their entirety.

### SUMMARY OF THE INVENTION

In some aspects, this disclosure provides for a method for the preparation of a highly pure triethylenetetramine diacid salt comprising the steps of:
a) contacting impure triethylenetetramine (TET) with a diacid resulting in a crude solid; and
b) recrystallizing the crude solid to obtain a highly pure triethylenetetramine diacid salt.
   In some aspects, said highly pure triethylenetetramine diacid salt is between about 95% and about 99.95% pure. In some aspects, said highly pure triethylenetetramine diacid salt is between about 98% and about 99% pure. In some aspects, said highly pure triethylenetetramine diacid salt is about 98.5% pure. In some aspects, said highly pure triethylenetetramine diacid salt is highly pure triethylenetetramine disuccinate (TES). In some aspects, the diacid is selected from the group consisting of succinic acid, malonic acid, oxalic acid, maleic acid, fumaric acid, malic acid tartaric acid and derivatives thereof, dibenzoyl tartaric acid, glutaric acid and adipic acid. In some aspects, the diacid is succinic acid. In some aspects, said impure triethylenetetramine (TET) is between about 60% and about 95% pure. In some aspects, said impure triethylenetetramine (TET) is between about 60% and about 70% pure. In some aspects, said impure triethylenetetramine (TET) is between about 60% and about 65% pure.

In some aspects, this disclosure provides for a method of recrystallization, the method comprising the steps of:
a) dissolving or partially dissolving the crude solid in a suitable solvent at an elevated temperature resulting in a solution or suspension;
b) cooling the solution or suspension to form a highly pure triethyeletetramine diacid salt as a crystal; and
c) collecting the highly pure triethylenetetramine diacid salt.
   In some aspects, said suitable solvent is selected from the group consisting of polar protic and polar aprotic solvents or mixtures thereof. In some aspects, said polar protic solvent is selected from water, C₁-C₈ alcohols and C₁-C₈ polyalcohols. In some aspects, said alcohols are selected from the group consisting of methanol, ethanol, isopropanol, 1-propanol, ethylene glycol, propylene glycol and butanediol. In some aspects, said C₁-C₈ alcohol is methanol. In some aspects, said polar aprotic solvent is selected from the group consisting of C₁-C₈ ethers, cyclic C₄-C₆ ethers, acetone, 2-butanone, dimethylacetamide, N-methylpyrrolidinone, N, N-dimethylformamide, dimethyl sulfoxide, acetonitrile and dimethylpropylene urea, pyridine, sulfolane and hexamethylphosphoric triamide. In some aspects, said polar aprotic solvent is acetonitrile. In some aspects, said mixture is water and acetonitrile. In some aspects, said mixture is between about 2% and between about 25% water. In some aspects, said mixture is between about 5% and about 15% water. In some aspects, said mixture is water and a C₁-C₈ alcohol. In some aspects, said alcohol is selected from the group consisting of methanol, ethanol, isopropanol and 1-propanol. In some aspects, said alcohol is methanol. In some aspects, said mixture is between about 2% and about 25% water. In some aspects, said mixture is between about 5% and about 10% water. In some aspects, the elevated temperature is between about 20° C. and about the boiling point of the solvent or mixture of solvents.

In some aspects, this disclosure provides for a method for the preparation of a highly pure triethylenetetramine diacid salt comprising the steps of:
a) dissolving impure triethylenetetramine (TET) in a suitable solvent giving a first solution;
b) adding to the first solution a diacid to form a second mixture;
c) allowing the second mixture to form a precipitate of crude triethylenetetramine diacid salt;
d) collecting the crude triethylenetetramine diacid salt; and
e) optionally, recrystallizing the crude triethylenetetramine diacid salt to obtain highly pure triethylenetetramine diacid salt.
   In some aspects, said highly pure triethylenetetramine diacid salt is between about 95% and about 99.95% pure. In some aspects, said highly pure triethylenetetramine diacid salt is between about 98% and about 99% pure. In some aspects said highly pure triethylenetetramine diacid salt is about 98.5% pure. In some aspects, said highly pure triethylenetetramine diacid salt is highly pure triethylenetetramine succinate (TES). In some aspects, said diacid is selected from the group consisting of succinic acid, malonic acid, maleic acid, fumaric acid, malic acid, tartaric acid and derivatives thereof, dibenzoyltartaric acid, glutaric acid and adipic acid. In some aspects, the diacid is succinic acid. In some aspects, said impure triethylenetetramine (TET) is between about 60% and about 95% pure. In some aspects, said impure triethylenetetramine (TET) is between about 60% and about 70% pure. In some aspects, said impure triethylenetetramine (TET) is about 60% pure. In some aspects the solvent is selected from the group consisting of C₁-C₈ alcohols, water, acetonitrile and tetrahydrofuran or mixtures thereof.

In some aspects, the step of recrystallizing the crude triethylenetetramine diacid salt as needed to obtain highly pure triethylenetetramine diacid salt is performed by the method comprising the steps of:
i) dissolving or partially dissolving the crude solid in a suitable solvent at an elevated temperature resulting in a solution;
ii) cooling the solution to form a highly pure triethylenetetramine diacid salt as a crystal; and
iii) collecting the highly pure triethylenetetramine diacid salt.
   In some aspects, said suitable solvent is selected from the group consisting of polar protic and polar aprotic solvents or mixtures thereof. In some aspects, said polar protic solvent is selected from water, C₁-C₈ alcohols and C₁-C₈ polyalcohols. In some aspects, said alcohols are selected from the group consisting of methanol, ethanol, isopropanol, 1-propanol ethylene glycol, propylene glycol and butanediol. In some aspects, said C₁-C₈ alcohol is methanol. In some aspects, said polar aprotic solvent is selected from the group consisting of C₁-C₈ ethers, cyclic C₄-C₆ ethers, acetone, 2-butanone, dimethylacetamide, N-methylpyrrolidinone, N,N-dimethylformamide, dimethyl sulfoxide, acetonitrile, dimethylpropylene urea, pyridine, sulfolane and hexamethylphosphoric triamide. In some aspects, said polar aprotic solvent is acetonitrile. In some aspects, said mixture is water and acetonitrile. In some aspects, said mixture is between about 2% and between about 25% water. In some aspects, said mixture is between about 5% and about 10% water. In some aspects, said mixture is water and a C₁-C₈ alcohol. In some aspects, said alcohol is selected from the group consisting of methanol, ethanol, isopropanol and 1-propanol. In some aspects, said alcohol is methanol. In some aspects, said mixture is between about 2% and about 25% water. In some aspects, said mixture is between about 5% and about 15% water. In some aspects, the elevated temperature is between about 20° C. and about the boiling point of the solvent or mixture of solvents.

In some aspects, this disclosure provides for a method for the preparation of a highly pure triethylenetetramine diacid salt comprising the steps of
a) dissolving impure triethylenetetramine (TET) in a first solvent giving a first solution;
b) warming the first solution to a first temperature;
c) adding a diacid as a solid or in a solution in a second solvent to the first solution at a first temperature to form a second solution;
d) optionally, cooling the second solution to a second temperature to form a precipitate of highly pure triethylenetetramine diacid salt; and
e) collecting the highly pure triethylenetetramine diacid.
   In some aspects, said highly pure triethylenetetramine diacid salt is between about 95% and about 99.95% pure. In some aspects, said highly pure triethylenetetramine diacid salt is between about 98% and about 99% pure. In some aspects, said highly pure triethylenetetramine diacid salt is about 98.5% pure. In some aspects, said highly pure triethylenetetramine diacid salt is highly pure triethylenetetramine disuccinate (TES). In some aspects, said diacid is selected from the group consisting of succinic acid, malonic acid, maleic acid, fumaric acid, malic acid, tartaric acid and derivatives thereof, dibenzoyl tartaric acid, glutaric acid and adipic acid. In some aspects, the diacid is succinic acid. In some aspects, said impure triethylenetetramine (TET) is between about 60% and about 90% pure. In some aspects, said impure triethylenetetramine (TET) is between about 60% and about 70% pure. In some aspects, said impure triethylenetetramine (TET) is about 65% pure. In some aspects, said first solvent is selected from the group consisting of C₁-C₈ alcohols, C₁-C₈ poly alcohols, acetonitrile, tetrahydrofuran and water and mixtures thereof. In some aspects, said alcohol is selected from the group consisting of methanol, ethanol, isopropanol, 1-propanol ethylene glycol, propylene glycol and butanediol. In some aspects, said first temperature is between about 50° C. and about 95° C. In some aspects, said first temperature is between about 65° C. and about 80° C. In some aspects, said second solvent is selected from the group consisting of C₁-C₈ alcohols, acetonitrile, tetrahydrofuran and water and mixtures thereof. In some aspects, said second temperature is between about −5° C. and about 25° C. In some aspects, said second temperature is about 0° C.

In some aspects, this disclosure provides for a method for the preparation of a highly pure triethylenetetramine dihydrochloride salt or highly pure triethylenetetramine tetrahydrochloride salt from impure triethylenetetramine comprising the steps of:
a) contacting impure triethylenetetramine (TET) with a diacid resulting in a crude solid;
b) recrystallizing the crude solid to obtain a highly pure triethylenetetramine diacid salt;
c) contacting the highly pure triethylenetetramine diacid salt with aqueous base to form highly pure triethylenetetramine (TET); and
d) converting the highly pure triethylenetetramine (TET) into its dihydrochloride salt by the methods described herein.
   In some aspects, said highly pure triethylenetetramine diacid salt is between about 95% and about 99.95% pure. In some aspects, said highly pure triethylenetetramine diacid salt is between about 98% and about 99% pure. In some aspects, said highly pure triethylenetetramine diacid salt is about 98.5% pure. In some aspects, said highly pure triethylenetetramine diacid salt is highly pure triethylenetetramine disuccinate (TES). In some aspects, said diacid is selected from the group consisting of succinic acid, malonic acid, oxalic acid, maleic acid, fumaric acid, malic acid, tartaric acid and derivatives thereof, dibenzoyl tartaric acid, glutaric acid and adipic acid. In some aspects, the diacid is succinic acid. In some aspects, the aqueous base is selected from the group consisting of aqueous sodium hydroxide, aqueous potassium hydroxide, aqueous sodium carbonate and aqueous potassium carbonate. In some aspects, said impure triethylenetetramine (TET) is between about 60% and about 90% pure. In some aspects, said impure triethylenetetramine (TET) is between about 60% and about 70% pure. In some aspects, said impure triethylenetetramine (TET) is about 60% pure.

In some aspects, this disclosure provides for a recrystallization method, wherein the recrystallization method is performed by a method comprising the following:
i) dissolving or partially dissolving the crude solid in a suitable solvent at an elevated temperature resulting in a solution or suspension;
i) cooling the solution or suspension to form highly pure triethylenetetramine diacid salt as a solid crystal; and
iii) collecting the highly pure triethylenetetramine diacid salt.
   In some aspects, said suitable solvent is selected from the group consisting of polar protic and polar aprotic solvents or mixtures thereof. In some aspects, said polar protic solvent is selected from water, C₁-C₈ alcohols and C₁-C₈ poly alcohols. In some aspects, said alcohol is selected from the group consisting of methanol, ethanol, isopropanol, 1-propanol, ethylene glycol, propylene glycol and butanediol. In some aspects, said C₁-C₈ alcohol is methanol. In some aspects, said polar aprotic solvent is selected from the group consisting of C₁-C₈ ethers, cyclic C₄-C₆ ethers, acetone, 2-butanone, dimethylacetamide, N-methylpyrrolidinone, N,N-dimethylformamide, dimethyl sulfoxide, acetonitrile dimethylpropylene urea, pyridine, sulfolane and hexamethylphosphoric triamide. In some aspects, said polar aprotic solvent is acetonitrile. In some aspects, said mixture is water and acetonitrile. In some aspects, said mixture is between about 2% and between about 25% water. In some aspects, said mixture is between about 5% and about 10% water. In some aspects, said mixture is water and a C₁-C₈ alcohol. In some aspects, said alcohol is selected from the group consisting of methanol, ethanol, isopropanol and 1-propanol. In some aspects, said alcohol is methanol. In some aspects, said mixture is between about 2% and about 25% water. In some aspects, said mixture is between about 5% and about 10% water. In some aspects, the elevated temperature is between about 20° C. and about the boiling point of the solvent or mixture of solvents.

In some aspects, this disclosure provides for a method for the preparation of highly pure triethylenetetramine (TET) by contacting the highly pure triethylenetetramine diacid salt with an aqueous base solution to form highly pure triethylenetetramine.

In some aspects, this disclosure provides for a method for the preparation of highly pure triethylenetetramine (TET) by contacting the highly pure triethylenetetramine diacid salt with a material capable of performing an ion exchange such as an ion exchange resin to form highly pure triethylenetetramine.

### DETAILED DESCRIPTION OF THE INVENTION

When describing the compounds and processes of the invention, the terms used are to be construed in accordance with the following definitions, unless a context dictates otherwise. The term “about” as used herein when referring to a measurable value such as a parameter, a purity, an amount, a temporal duration, and the like, is meant to encompass variations of +/−10% or less, preferably +1-5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Reference processes to make the various salts of triethylenetetramine require substantially pure triethylenetetramine (about 98% or greater) which is difficult and expensive to produce. Technical grade triethylenetetramine would be a convenient and cost-effective starting material in the synthesis of its salts, however, it is typically contaminated with impurities such as ethylenediamine, diethylenetriamine, piperazine and various derivatives, and branched polyamine impurities making it unsuitable to produce a single pure salt.

In some embodiments, this disclosure provides cost-effective ways to produce not only highly pure (98% or greater) triethylenetetramine but also the various salts of triethylenetetramine. Highly pure (about 98% or greater) triethylenetetramine can be produced from inexpensive technical grade (about 65% pure; about $30 USD per Kg) triethylenetetramine, and optionally converted into its salt forms. For example, the highly pure triethylenetetramine so produced may be converted into its dihydrochloride salt using any of the methods described herein. In some embodiments, this disclosure provides for a commercially practicable and cost-effective method to produce highly pure crystalline diacid salts of triethylenetetramine, including a highly pure crystalline disuccinate salt starting from technical grade triethylenetetramine. Technical grade (e.g. impure) triethylenetetramine (TET) may be obtained from the reaction of 1,2 diaminoethane reacted with ethanolamine and a catalyst.

The purification methods described herein offer a convenient, cost effective, and industrially viable process for synthesis of triethylenetetramine dihydrochloride (1) which avoids the following. a) lengthy synthetic routes for protection and deprotection of the reactant amines and intermediates, b) excessive use of organic solvents, c) use of mineral acids in multiple steps. Another advantage of the purification methods of this disclosure are the avoidance of strong bases, a controlled number of unit operations, and good yield of the desired dihydrochloride.

As used herein, the term “salt” or “pharmaceutically acceptable salt” refers to a salt derived from pharmaceutically compatible organic and/or inorganic counter-ions of the compound referred to.

As used herein, the term “Cx-y” when used in conjunction with a chemical moiety, such as alkyl, alkenyl, or alkynyl is intended to include groups that contain from x to y carbons in the chain. For example, the term “Cx-y alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. The term “Cx-y alcohol” refers to substituted or unsubstituted saturated aliphatic alcohol groups, including straight-chain alkyl alcohol and branched-chain alkyl alcohol groups that contain from x to y carbons in the alkyl chain.

Alcohols and/or alcohol solvents are typically short chain alcohols. Alcohols typically are of formula ROH, wherein R is a straight or branched C₁₋₈ alkyl group. The C₁₋₈ alkyl group is preferably unsubstituted. Examples of alcohols include methanol, ethanol, n-propanol, propanol, n-butanol, i-butanol, s-butanol and t-butanol. Methanol and ethanol are preferred. Methanol is more preferred.

Ethers and/or ether solvents are typically short chain ethers. Ethers typically are of formula R—O—R′, wherein R and R′ are the same or different and represent a straight or branched C₁₋₈ alkyl group. The C₁₋₈ alkyl group is preferably unsubstituted. Preferred ethers include diethylether, diisopropylether, and methyl t-butyl ether (MTBE). C₄₋₆ cyclic ethers are also included such as tetrahydrofuran and tetrahydropyran. Nitrile solvents are well known to the person skilled in the art. Nitriles are typically short chain nitriles. Nitriles typically are of formula R—CN, wherein R represents a straight or branched C₁₋₆ alkyl group. The C₁₋₆ alkyl group is preferably unsubstituted. Preferred nitriles include acetonitrile.

As used herein, the term “aqueous” when referring to an aqueous organic solvent refers to a composition comprising water and the recited organic compound. For example, “aqueous methanol” refers to a composition comprising from 1 to 99 wt. % water with the remainder methanol. In some embodiments, the water phase can further comprise dissolved salts.

In some embodiments, the aqueous organic solvent is aqueous alcohol or aqueous acetonitrile. The aqueous organic solvent is preferably aqueous methanol or aqueous acetonitrile.

The purity of the reagents and products can be determined by any of the analytical methods known to those skilled in the art such as ion chromatography, high-pressure liquid chromatography (HPLC), proton nuclear magnetic resonance spectroscopy (¹H NMR), carbon nuclear magnetic resonance spectroscopy), ¹³C NMR, infrared spectroscopy (IR), powder X-ray diffraction (PXRD), differential scanning calorimetry (DSC) and melting point.

In some embodiments, this disclosure relates to a unit dose of a pharmaceutical composition for treatment of a disease involving a copper-mediated pathway,
wherein the unit dose contains an effective amount of a TET salt formulated as a pharmaceutical composition for administration to a subject in need thereof, wherein the formulation of the composition and the amount of the TET salt in the unit dose configures the unit dose to be effective in treating the disease involving a copper-mediated pathway by removing or reducing the amount of levels of free copper (e.g., copper(I) or copper(II)) from the blood or tissue of said subject, thereby decreasing the severity of one or more signs or symptoms of the disease involving a copper-mediated pathway without causing adverse effects when administered to the subject as a single dose,
wherein the TET salt has been prepared by a synthesis method that includes the steps of:
   i) dissolving impure triethylenetetramine in a polar protic or polar aprotic solvent to form dissolved impure triethylenetetramine solution at a first temperature;
   ii) optionally dissolving succinic acid in a protic or polar aprotic solvent to form dissolved succinic acid solution at a second temperature;
   iii) adding optionally solid succinic acid or optionally adding the dissolved succinic acid solution at a third temperature to form a dissolved triethylenetetramine succinate solution;
   iv) allowing the dissolved triethylenetetramine succinate solution to cool to a forth temperature whereby solid triethylenetetramine succinate (TES) forms; and
   v) collecting said solid triethylenetetramine succinate (TES),
   wherein the first temperature ranges from about 50° C. to about 70° C.,
   wherein the second temperature ranges from about 50° C. to about 70° C.,
   wherein the third temperature ranges from about 50° C. to about 70° C., and
   wherein the fourth temperature ranges from about 0° C. to about 40° C.
      In some embodiments, the polar protic solvent is methanol. In some embodiments, the polar aprotic solvent is acetonitrile. In some embodiments the polar protic solvent will be a mixture of water and an alcohol such as methanol, ethanol, or 2-propanol.

Certain Methods involving Ion Exchange

In some embodiments, this disclosure provides for a method for the preparation of highly pure triethylenetetramine (TET) by contacting the highly pure triethylenetetramine diacid salt with a material capable of performing an ion exchange to form highly pure triethylenetetramine. The material capable of performing an ion exchange can be an ion exchange resin. The ion exchange resin can be cationic or anionic. In some embodiments, the method of preparing highly pure TET by a process involving ion exchange resins involves contacting the TET salt with the acid form of the ion exchange resin, then filtering the ion exchange resin from the solution after a selected (e.g., 1 minute to 8 hours) of time, at room temperature. In some embodiments, the ion exchange method for highly pure TET preparation can be similar to those methods described in U.S. Pat. No. 4,263,145, herein incorporated by reference.

### EXAMPLES

### Example 1

7.30 grams (30 mmol corrected for purity) of technical grade (60% purity; Mallinckrodt Sigma-Aldrich) triethylenetetramine was dissolved in 50 mL of acetonitrile and warmed to 65° C. with stirring. Succinic acid (11.8 gm, 100 mmol) was separately dissolved in a warm (65° C.) solution of 75 mL acetonitrile:water (2:1). The warm solution of succinic acid was added to the acetonitrile solution of triethylenetetramine over approximately 3 hours. After addition was complete, the solution was cooled to room temperature over approximately 4 hours. The formation of a crystalline precipitate was seen to spontaneously occur at between 40-45° C. Alternatively, crystallization may be initiated by seeding with a small amount (less than 500 mg) of preformed crystals at approximately 55° C. or lower temperature. After cooling to 20° C., the suspension was stirred for another 2 hours before filtration of the crystalline solids. The solids were washed with 30 mL of acetonitrile and dried to a constant weight to obtain 10.11 grams (88.3%) of crystalline triethylenetetramine disuccinate which was identical (PXRD, single crystal X-ray, 1H and 13C NMR) to reference material. (U.S. Pat. No. 9,556,123 incorporated by reference in its entirety)

### Example 2

7.30 grams (48.5 mmol corrected for purity) of 97% purity triethylenetetramine (Mallinckrodt Sigma-Aldrich) was dissolved in 50 mL of acetonitrile and warmed to 65° C. with stirring Succinic acid (11.8 gm, 100 mmol) separately dissolved in a warm (65° C.) solution of 75 acetonitrile:water (2:1). The warm solution of succinic acid was added to the acetonitrile solution of triethylenetetramine over approximately 3 hours. After addition was complete, the solution was cooled to room temperature over approximately 4 hours. The formation of a second, oily layer was observed to occur spontaneously at approximately 50° C. This layer appeared to be a mixture of crystalline and oily material. With continued stirred at 20° C. (approximately 2 hours), the precipitated material was entirely converted into a heavy mass of crystalline material. The solids were isolated by filtration and washed with 30 mL of acetonitrile. After drying to a constant weight, 17.09 grams of crystalline triethylenetetramine disuccinate was obtained. The product was identical (PXRD, 1H and 13C NMR) with material in the prior art.

### Example 3

The process of Example 1 was repeated (all weights and volumes of materials and solvents being identical), with the exception of using methanol rather than acetonitrile as the solvent. Using 60% purity triethylenetetramine (as in Example 1) resulted in the isolation of 9.51 grams (83.0% yield corrected for purity) of crystalline triethylenetetramine disuccinate salt.

### Example 4

7.30 grams (48.5 mmol corrected for purity) of 97% purity triethylenetetramine (Mallinckrodt Sigma-Aldrich), was dissolved in 50 mL of methanol and stirred at 25° C. A solution of succinic acid (11.8 gm, 100 mmol) in 100 mL of 60° C. methanol was added to the solution over 30 minutes. After about 25% of the succinic acid solution had been added, the precipitation of a second, oily layer was observed. After addition was complete, the suspension was cooled to 20° C. over about 5 hours and stirred for an additional 18 hours. At this time, the conversion of the oily layer into a crystalline precipitate was noted. The product was isolated by filtration and washing with 75 mL of methanol. After drying to a constant weight, 17.27 grams (93.2% yield) of crystalline product was obtained, identical in all respects with material obtained from Examples 1-3.

### Example 5

7.30 grams (30 mmol corrected for purity) of 60% purity triethylenetetramine (Mallinckrodt Sigma-Aldrich), was dissolved in 50 mL of methanol and stirred at 25° C. A solution of succinic acid (11.8 gm, 100 mmol) in 100 mL of 60° C. methanol was added to the solution over 30 minutes. After about 25% of the succinic acid solution had been added, the precipitation of a second, oily layer was observed. After addition was complete, the suspension was cooled to 20° C. over about 5 hours and stirred for an additional 18 hours. At this time, the conversion of the oily layer into a crystalline precipitate was noted. The product was isolated by filtration and washing with 50 mL of methanol. After drying to a constant weight, 9.27 grams (80.9% yield) of crystalline product was obtained, identical in with material obtained from Examples 1-4.

### Example 6

7.30 grams (48.5 mmol corrected for purity) of 97% purity triethylenetetramine (Mallinckrodt Sigma-Aldrich), was dissolved in 50 mL of acetonitrile and stirred at 25° C. A solution of succinic acid (11.8 gm, 100 mmol) in 100 mL of 70° C. acetonitrile was added to the solution over 30 minutes. After about 25% of the succinic acid solution had been added, the precipitation of a second, oily layer was observed. After addition was complete, the suspension was cooled to 20° C. over about 5 hours and stirred for an additional 18 hours. At this time, the conversion of the oily layer into a crystalline precipitate was noted. The product was isolated by filtration and washing with 75 mL of acetonitrile. After drying to a constant weight, 14.89 grams (80.4% yield) of crystalline product was obtained, identical in all respects with material obtained from other examples.

### Example 7

7.30 grams (30 mmol corrected for purity) of 60% purity triethylenetetramine (Mallinckrodt Sigma-Aldrich), was dissolved in 50 mL of methanol and stirred at 25° C. A solution of succinic acid (7.08 gm, 60 mmol) in 100 mL of 60° C. methanol was added to the solution over 30 minutes. After about 50% of the succinic acid solution had been added, the precipitation of a second, oily layer was observed. After addition was complete, the suspension was cooled to 20° C. over about 5 hours and stirred for an additional 18 hours. At this time, the conversion of the oily layer into a crystalline precipitate was noted. The product was isolated by filtration and washing with 50 mL of methanol. After drying to a constant weight, 8.47 grams (73.9% yield) of crystalline product was obtained, identical in with material obtained from other examples.

In an additional embodiment of the invention, crystalline salts of triethylenetetramine and organic diacids can be recrystallized to improve their purity. Thus, triethylenetetramine disuccinate salt of 97.6% purity can be recrystallized as follows to improve the purity to greater than 99%.

### Example 8

Triethylenetetramine disuccinate salt (7.64 grams, approximately 20 mmol) of 97.6% purity (determined by GC/MS) was dissolved with heating and stirring in 120 mL of 10% aqueous methanol to yield a clear solution at 60° C. The solution was slowly cooled over about 2 hours to 20° C. and stirring was maintained for an additional 1 hour at this temperature. The solution was further cooled to approximately 5° C. and held at this temperature for an additional 2 hours. The solution was filtered, and the filter cake was washed with approximately 40 mL of methanol. The filter cake was dried to a constant weight at 50° C. under reduced pressure. The product was obtained in a purity of 99.6% (GC/MS) in a yield of 6.30 grams (84% recovery).

In an additional embodiment of the invention, crystalline salts of triethylenetetramine and organic diacids may be converted to the free amine in high recovery and high purity.

### Example 9

Triethylenetetramine disuccinate (3.82 grams, approximately 10 mmol) of 99.95% purity (determined by ion chromatography) was suspended in 40 mL of isopropyl acetate. The suspension was shaken with 20 mL of 3N aqueous sodium hydroxide to obtain a clear, two-phase liquid mixture. Solid sodium chloride was added to the aqueous layer to obtain a saturated solution. The layers were separated, and the aqueous layer was extracted twice with 30 mL portions of isopropyl acetate. The combined organic layers were washed with saturated aqueous brine (50 mL). The isopropyl acetate was removed under reduced pressure to provide 1.23 gm of triethylenetetramine in a purity of 99.2% (containing 0.4% isopropyl acetate; recovery of 83%).

### Example 10

Triethylenetetramine disuccinate (3.82 grams, approximately 10 mmol) of 99.95% purity (determined by ion chromatography) was suspended in 40 mL of isopropyl acetate. The suspension was shaken with 20 mL of 3N aqueous sodium hydroxide to obtain a clear, two-phase liquid mixture. The layers were separated, and the aqueous layer was extracted twice with 30 mL portions of isopropyl acetate. The combined organic layers were dried over solid sodium chloride and concentrated to a volume of approximately 3 mL. The crude product was purified by flash column chromatography using a Biotage® KP-NH column, with hexane/EtOAc (0%-30% ethyl acetate gradient) as the mobile phase. Triethylenetetramine was obtained in greater than 99% purity and approximately 75%-90% recovery after removal of the volatile solvent.

### Example 11

Triethylenetetramine disuccinate (3.82 grams, approximately 10 mmol) of 99.95% purity (determined by ion chromatography) was suspended in 40 mL of isopropyl acetate. The suspension was shaken with 20 mL of 3N aqueous sodium hydroxide to obtain a clear, two-phase liquid mixture. The layers were separated, and the aqueous layer was extracted twice with 30 mL portions of isopropyl acetate. The combined organic layers were dried over solid sodium chloride and concentrated under reduced pressure to a volume of approximately 3 mL. The crude product was distilled using a kugelrohr apparatus. Triethylenetetramine was obtained in greater than 99% purity and approximately 81% recovery.

### Example 12

Triethylenetetramine disuccinate (3.82 grams, approximately 10 mmol) of 99.95% purity (determined by ion chromatography) was converted to the corresponding free amine by ion exchange chromatography using an anion exchange resin. After removal of solvent, the free triethylenetetramine was obtained in nearly quantitative recovery.

### Additional Embodiments

Additional embodiments of this disclosure are provided as follows:
Embodiment 1. A method for the preparation of a highly pure triethylenetetramine diacid salt comprising the steps of
   a) contacting impure triethylenetetramine (TET) with a diacid resulting in a crude solid; and
   b) recrystallizing the crude solid to obtain a highly pure triethylenetetramine diacid salt.
Embodiment 2. The method according to embodiment 1 wherein said highly pure triethylenetetramine diacid salt is between about 95% and about 99.95% pure.
Embodiment 3. The method according to embodiment 2 wherein said highly pure triethylenetetramine diacid salt is between about 98% and about 99% pure.
Embodiment 4. The method according to embodiment 3 wherein said highly pure triethylenetetramine diacid salt is about 98.5% pure.
Embodiment 5. The method according to embodiments 1, 2, 3 or 4 wherein said highly pure triethylenetetramine diacid salt is highly pure triethylenetetramine disuccinate (TES).
Embodiment 6. The method according to embodiment 1 wherein said diacid is selected from the group consisting of succinic acid, malonic acid, oxalic acid, maleic acid, fumaric acid, malic acid, tartaric acid and derivatives thereof, dibenzoyl tartaric acid, glutaric acid and adipic acid.
Embodiment 7. The method according to embodiment 6 wherein the diacid is succinic acid.
Embodiment 8. The method according to embodiment 1 wherein said impure triethylenetetramine (TET) is between about 60% and about 95% pure.
Embodiment 9. The method according to embodiment 8 wherein said impure triethylenetetramine (TET) is between about 60% and about 70% pure.
Embodiment 10. The method according to embodiment 9 wherein said impure triethylenetetramine (TET) is between about 60% and about 65% pure.
Embodiment 11. The method according to embodiment 1 wherein said recrystallization is accomplished by
   a) dissolving or partially dissolving the crude solid in a suitable solvent at an elevated temperature resulting in a solution or suspension;
   b) cooling the solution or suspension; and
   c) collecting highly pure triethylenetetramine diacid salt.
Embodiment 12. The method according to embodiment 11 wherein said suitable solvent is selected from the group consisting of polar protic solvent, polar aprotic solvent, or mixtures thereof.
Embodiment 13. The method according to embodiment 12 wherein said polar protic solvent is selected from water, C₁-C₈ alcohols and C₁-C₈ polyalcohols.
Embodiment 14. The method according to embodiment 13 wherein said C₁-C₈ alcohols are selected from the group consisting of methanol, ethanol, isopropanol, 1-propanol ethtlene glycol, propylene glycol and butanediol.
Embodiment 15. The method according to embodiment 14 wherein said C₁-C₈ alcohol is methanol.
Embodiment 16. The method according to embodiment 12 wherein said polar aprotic solvent is selected from the group consisting of C₁-C₈ ethers, cyclic C₄-C₆ ethers, acetone, 2-butanone, dimethylacetamide, N-methylpyrrolidinone, N,N-dimethylformamide, dimethyl sulfoxide, acetonitrile, dimethylpropylene urea, pyridine, sulfolane and hexamethylphosphoric triamide.
Embodiment 17. The method according to embodiment 16 wherein said polar aprotic solvent is acetonitrile.
Embodiment 18. The method according to embodiment 17 wherein said mixture is water and acetonitrile.
Embodiment 19. The method according to embodiment 18 wherein said mixture is between about 2% and between about 25% water.
Embodiment 20. The method according to embodiment 19 wherein said mixture is between about 5% and about 15% water.
Embodiment 21. The method according to embodiment 12 wherein said mixture is water and a C₁-C₈ alcohol.
Embodiment 22. The method according to embodiment 21 wherein said alcohol is selected from the group consisting of methanol, ethanol, isopropanol and 1-propanol.
Embodiment 23. The method according to embodiment 22 wherein said alcohol is methanol.
Embodiment 24. The method according to embodiment 21 wherein said mixture is between about 2% and about 50% water.
Embodiment 25. The method according to embodiment 24 wherein said mixture is between about 5% and about 20% water.
Embodiment 26. The method according to embodiment 12 wherein the elevated temperature is between about 20° C. and about the boiling point of the solvent or mixture of solvents.
Embodiment 27. A method for the preparation of a highly pure triethylenetetramine diacid salt comprising the steps of
   a) dissolving impure triethylenetetramine (TET) in a suitable solvent giving a first solution;
   b) adding to the first solution a diacid to form a second mixture;
   c) allowing to stand or stirring the second mixture to form a precipitate of crude triethylenetetramine diacid salt;
   d) collecting the crude triethylenetetramine diacid salt; and
   e) optionally, recrystallizing the crude triethylenetetramine diacid salt as needed to obtain highly pure triethylenetetramine diacid salt.
Embodiment 28. The method according to embodiment 24 wherein said highly pure triethylenetetramine diacid salt is between about 95% and about 99.95% pure.
Embodiment 29. The method according to embodiment 25 wherein said highly pure triethylenetetramine diacid salt is between about 98% and about 99% pure.
Embodiment 30. The method according to embodiment 26 wherein said highly pure triethylenetetramine diacid salt is about 98.5% pure.
Embodiment 31. The method according to embodiments 27, 28, 29 or 30 wherein said highly pure triethylenetetramine diacid salt is highly pure triethylenetetramine disuccinate (TES).
Embodiment 32. The method according to embodiment 27 wherein said diacid is selected from the group consisting of succinic acid, malonic acid maleic acid, fumaric acid, malic acid, tartaric acid and derivatives thereof, dibenzoyltartaric acid, glutaric acid and adipic acid.
Embodiment 33. The method according to embodiment 32 wherein the diacid is succinic acid.
Embodiment 34. The method according to embodiment 27 wherein said impure triethylenetetramine (TET) is between about 60% and about 95% pure.
Embodiment 35. The method according to embodiment 34 wherein said impure triethylenetetramine (TET) is between about 60% and about 70% pure.
Embodiment 36. The method according to embodiment 25 wherein said impure triethylenetetramine (TET) is about 60% pure.
Embodiment 37. The method according to embodiment 27 wherein the solvent is selected from the group consisting of C₁-C₈ alcohols, water, acetonitrile and tetrahydrofuran or mixtures thereof.
Embodiment 38. The method according to embodiment 27 wherein said recrystallization is performed by
   a) dissolving or partially dissolving the crude solid in a suitable solvent at an elevated temperature resulting in a solution;
   b) cooling the solution; and
   c) collecting highly pure triethylenetetramine diacid salt.
Embodiment 39. The method according to embodiment 38 wherein said suitable solvent is selected from the group consisting of a polar protic solvent, a polar aprotic solvent, or mixtures thereof.
Embodiment 40. The method according to embodiment 39 wherein said polar protic solvent is selected from water, C₁-C₈ alcohols and C₁-C₈ poly alcohols.
Embodiment 41. The method according to embodiment 40 wherein said alcohols are selected from the group consisting of methanol, ethanol, isopropanol, 1-propanol, ethylene glycol, propylene glycol and butanediol.
Embodiment 42. The method according to embodiment 40 wherein said C₁-C₈ alcohols is methanol.
Embodiment 43. The method according to embodiment 39 wherein said polar aprotic solvent is selected from the group consisting of C₁-C₈ ethers, cyclic C₄-C₆ ethers, acetone, 2-butanone, dimethylacetamide, N-methylpyrrolidinone, N, N-dimethylformamide, dimethyl sulfoxide, acetonitrile dimethylpropylene urea, pyridine, sulfolane and hexamethylphosphoric triamide.
Embodiment 44. The method according to embodiment 43 wherein said polar aprotic solvent is acetonitrile.
Embodiment 45. The method according to embodiment 39 wherein said mixture is water and acetonitrile.
Embodiment 46. The method according to embodiment 45 wherein said mixture is between about 2% and between about 50% water.
Embodiment 47. The method according to embodiment 46 wherein said mixture is between about 5% and about 20% water.
Embodiment 48. The method according to embodiment 45 wherein said mixture is water and a C₁-C₈ alcohol.
Embodiment 49. The method according to embodiment 48 wherein said alcohol is selected from the group consisting of methanol, ethanol, isopropanol and 1-propanol.
Embodiment 50. The method according to embodiment 49 wherein said alcohol is methanol.
Embodiment 51. The method according to embodiment 48 wherein said mixture is between about 2% and about 50% water.
Embodiment 52. The method according to embodiment 51 wherein said mixture is between about 5% and about 20% water.
Embodiment 53. The method according to embodiment 38 wherein the elevated temperature is between about 20° C. and about the boiling point of the solvent or mixture of solvents.
Embodiment 54. A method for the preparation of a highly pure triethylenetetramine diacid salt comprising the steps of
   a) dissolving impure triethylenetetramine (TET) in a first solvent giving a first solution;
   b) warming the first solution to a first temperature;
   c) adding a diacid as a solid or in a solution in a second solvent to the first solution at a first temperature to form a second solution;
   d) optionally cooling the second solution to a second temperature to form a precipitate of highly pure triethylenetetramine diacid salt; and
   e) collecting the highly pure triethylenetetramine diacid salt.
Embodiment 55. The method according to embodiment 54 wherein said highly pure triethylenetetramine diacid salt is between about 95% and about 99.95% pure.
Embodiment 56. The method according to embodiment 55 wherein said highly pure triethylenetetramine diacid salt is between about 98% and about 99% pure.
Embodiment 57. The method according to embodiment 56 wherein said highly pure triethylenetetramine diacid salt is about 98.5% pure.
Embodiment 58. The method according to embodiments 54, 55, 56 or 57 wherein said highly pure triethylenetetramine diacid salt is highly pure triethylenetetramine disuccinate (TES).
Embodiment 59. The method according to embodiment 54 wherein said diacid is selected from the group consisting of succinic acid, malonic acid, fumaric acid, malic acid, maleic acid, tartaric acid and derivatives thereof, dibenzoyl tartaric acid, oxalic acid, glutaric acid and adipic acid.
Embodiment 60. The method according to embodiment 59 wherein the diacid is succinic acid.
Embodiment 61. The method according to embodiment 54 wherein said impure triethylenetetramine (TET) is between about 60% and about 90% pure.
Embodiment 62. The method according to embodiment 61 wherein said impure triethylenetetramine (TET) is between about 60% and about 70% pure.
Embodiment 63. The method according to embodiment 62 wherein said impure triethylenetetramine (TET) is about 60% pure.
Embodiment 64. The method according to embodiment 63 wherein said first solvent is selected from the group consisting of C₁-C₈ alcohols, C₁-C₈ poly alcohols, acetonitrile, tetrahydrofuran and water and mixtures thereof.
Embodiment 65. The method according to embodiment 64 wherein said alcohol is selected from the group consisting of methanol, ethanol, isopropanol, 1-propanol ethylene glycol, propylene glycol and butanediol.
Embodiment 66. The method according to embodiment 54 wherein said first temperature is between about 50° C. and about 95° C.
Embodiment 67. The method according to embodiment 66 wherein said first temperature is between about 65° C. and about 80° C.
Embodiment 68. The method according to embodiment 54 wherein said second solvent is selected from the group consisting of C₁-C₈ alcohols, acetonitrile, tetrahydrofuran and water and mixtures thereof.
Embodiment 69. The method according to embodiment 54 wherein said second temperature is between about −5° C. and about 25° C.
Embodiment 70. The method according to embodiment 60 wherein said second temperature is about 0° C.
Embodiment 71. A method for the preparation of a highly pure triethylenetetramine dihydrochloride salt comprising the steps of:
   a) contacting impure triethylenetetramine (TET) with a diacid resulting in a crude solid;
   b) recrystallizing the crude solid to obtain a highly pure triethylenetetramine diacid salt;
   c) contacting the highly pure triethylenetetramine diacid salt with aqueous base to form highly pure triethylenetetramine (TET); and
   d) converting the highly pure triethylenetetramine (TET) into its dihydrochloride salt using any of the literature methods described in the specification.
Embodiment 72. The method according to embodiment 71 wherein said highly pure triethylenetetramine diacid salt is between about 95% and about 99.95% pure.
Embodiment 73. The method according to embodiment 72 wherein said highly pure triethylenetetramine diacid salt is between about 98% and about 99% pure.
Embodiment 74. The method according to embodiment 73 wherein said highly pure triethylenetetramine diacid salt is about 98.5% pure.
Embodiment 75. The method according to embodiments 71, 72, 73 or 74 wherein said highly pure triethylenetetramine diacid salt is highly pure triethylenetetramine disuccinate (TES).
Embodiment 76. The method according to embodiment 71 wherein said diacid is selected from the group consisting of succinic acid, malonic acid, maleic acid, fumaric acid, malic acid, tartaric acid and derivatives thereof, dibenzoyl tartaric acid, oxalic acid, glutaric acid and adipic acid.
Embodiment 77. The method according to embodiment 76 wherein the diacid is succinic acid.
Embodiment 78. The method according to embodiment 71 wherein the aqueous base is selected from the group consisting of aqueous sodium hydroxide, aqueous potassium hydroxide, aqueous sodium carbonate and aqueous potassium carbonate.
Embodiment 79. The method according to embodiment 71 wherein said impure triethylenetetramine (TET) is between about 60% and about 90% pure.
Embodiment 80. The method according to embodiment 79 wherein said impure triethylenetetramine (TET) is between about 60% and about 70% pure.
Embodiment 81. The method according to embodiment 80 wherein said impure triethylenetetramine (TET) is about 60% pure.
Embodiment 82. The method according to embodiment 71 wherein said recrystallization is performed by the following steps:
   a) dissolving or partially dissolving the crude solid in a suitable solvent at an elevated temperature resulting in a solution or suspension;
   b) cooling the solution or suspension; and
   c) collecting highly pure triethylenetetramine diacid salt.
Embodiment 83. The method according to embodiment 82 wherein said suitable solvent is selected from the group consisting of polar protic and polar aprotic solvents or mixtures thereof.
Embodiment 84. The method according to embodiment 83 wherein said polar protic solvent is selected from water, C₁-C₈ alcohols and C₁-C₈ poly alcohols.
Embodiment 85. The method according to embodiment 84 wherein said C₁-C₈ alcohols alcohol are selected from the group consisting of methanol, ethanol, isopropanol, 1-propanol, ethylene glycol, propylene glycol and butanediol.
Embodiment 86. The method according to embodiment 84 wherein said C₁-C₈ alcohol is methanol.
Embodiment 87. The method according to embodiment 83 wherein said polar aprotic solvent is selected from the group consisting of C₁-C₈ ethers, cyclic C₄-C₆ ethers, acetone, 2-butanone, dimethylacetamide, N-methylpyrrolidinone, N, N-dimethylformamide, dimethyl sulfoxide, acetonitrile dimethylpropylene urea, pyridine, sulfolane and hexamethylphosphoric triamide.
Embodiment 88. The method according to embodiment 87 wherein said polar aprotic solvent is acetonitrile.
Embodiment 89. The method according to embodiment 87 wherein said mixture is water and acetonitrile.
Embodiment 90. The method according to embodiment 89 wherein said mixture is between about 2% and between about 25% water.
Embodiment 91. The method according to embodiment 90 wherein said mixture is between about 5% and about 10% water.
Embodiment 92. The method according to embodiment 83 wherein said mixture is water and a C₁-C₈ alcohol.
Embodiment 93. The method according to embodiment 92 wherein said alcohol is selected from the group consisting of methanol, ethanol, isopropanol and 1-propanol.
Embodiment 94. The method according to embodiment 93 wherein said alcohol is methanol.
Embodiment 95. The method according to embodiment 92 wherein said mixture is between about 2% and about 25% water.
Embodiment 96. The method according to embodiment 95 wherein said mixture is between about 5% and about 10% water.
Embodiment 97. The method according to embodiment 96 wherein the elevated temperature is between about 20° C. and about the boiling point of the solvent or mixture of solvents.
Embodiment 98. A method for the preparation of highly pure triethylenetetramine succinate from impure triethylenetetramine, the method comprising:
   i) dissolving impure triethylenetetramine in a polar protic or polar aprotic solvent to form dissolved impure triethylenetetramine solution at a first temperature;
   ii) dissolving succinic acid in an aqueous acetonitrile solution to form dissolved succinic acid solution at a second temperature;
   iii) adding the dissolved impure triethylenetetramine solution to dissolved succinic acid solution at a third temperature to form a dissolved triethylenetetramine succinate solution;
   iv) allowing the dissolved triethylenetetramine succinate solution to cool to a forth temperature whereby solid triethylenetetramine succinate (TES) forms; and
   v) collecting said solid triethylenetetramine succinate (TES),
   wherein the first temperature ranges from about 60° C. to about 70° C.,
   wherein the second temperature ranges from about 60° C. to about 70° C.,
   wherein the third temperature ranges from about 60° C. to about 70° C., and
   wherein the fourth temperature ranges from about 0° C. to about 25° C.
Embodiment 99. The method of embodiment 98, wherein the polar protic solvent is methanol.
Embodiment 100. The method of embodiment 98, wherein the polar aprotic solvent is acetonitrile.

What is claimed is:

1. A method for the preparation of a highly pure triethylenetetramine diacid salt comprising the steps of
a) contacting impure triethylenetetramine (TET) with a diacid resulting in a crude solid; and
b) recrystallizing the crude solid to obtain a highly pure triethylenetetramine diacid salt,
wherein the highly pure triethylenetetramine diacid salt is between about 95% and about 99.95% pure.

2. The method according to claim 1, wherein said highly pure triethylenetetramine diacid salt is about 98.5% pure.

3. The method according to claim 1 wherein said diacid is selected from the group consisting of succinic acid, malonic acid, oxalic acid, maleic acid, fumaric acid, malic acid, tartaric acid and derivatives thereof, dibenzoyl tartaric acid, glutaric acid and adipic acid.

4. The method according to claim 3, wherein the diacid is succinic acid.

5. The method according to claim 1, wherein the impure triethylenetetramine (TET) is between about 60% and about 70% pure.

6. The method according to claim 1, wherein said recrystallization is performed by a method comprising the steps of:
i) dissolving or partially dissolving the crude solid in a solvent selected from a polar aprotic solvent or a polar protic solvent at an elevated temperature resulting in a solution or suspension;
ii) cooling the solution or suspension; and
iii) collecting highly pure triethylenetetramine diacid salt.

7. The method according to claim 6, wherein said polar protic solvent is selected from water, C₁-C₈ alcohols, C₁-C₈ polyalcohols, and mixtures thereof.

8. The method according to claim 7, wherein said C₁-C₈ alcohols are selected from the group consisting of methanol, ethanol, isopropanol, 1-propanol ethtlene glycol, propylene glycol and butanediol.

9. The method according to claim 6, wherein said polar protic solvent is methanol.

10. The method according to claim 6, wherein said polar aprotic solvent is selected from the group consisting of C₁-C₈ ethers, cyclic C₄-C₆ ethers, acetone, 2-butanone, dimethylacetamide, N-methylpyrrolidinone, N,N-dimethylformamide, dimethyl sulfoxide, acetonitrile, dimethylpropylene urea, pyridine, sulfolane, hexamethylphosphoric triamide, and mixtures thereof.

11. The method according to claim 6, wherein said polar aprotic solvent is an aqueous solution comprising acetonitrile.

12. The method according to claim 6, wherein said aprotic solvent is acetonitrile.

13. The method according to claim 11, wherein said the aqueous solution comprising acetonitrile comprises between about 5% and between about 50% water.

14. The method according to claim 6, wherein the elevated temperature is between about ° C. and about the boiling point of the solvent or mixture of solvents.

15. A method for the preparation of a highly pure triethylenetetramine diacid salt from impure triethylenetetramine comprising the steps of:
a) dissolving impure triethylenetetramine (TET) in a suitable solvent giving a first solution;
b) adding to the first solution a diacid to form a second mixture;
c) allowing to stand or stirring the second mixture to form a precipitate of crude triethylenetetramine diacid salt;
d) collecting the crude triethylenetetramine diacid salt; and
e) optionally, recrystallizing the crude triethylenetetramine diacid salt to obtain highly pure triethylenetetramine diacid salt.

16. The method according to claim 15, wherein said highly pure triethylenetetramine diacid salt is between about 95% and about 99.95% pure.

17. The method according to claim 15, wherein said diacid is selected from the group consisting of succinic acid, malonic acid maleic acid, fumaric acid, malic acid, tartaric acid and derivatives thereof, dibenzoyltartaric acid, glutaric acid and adipic acid.

18. The method according to claim 17, wherein the diacid is succinic acid.

19. The method according to claim 15, wherein the impure triethylenetetramine (TET) is between about 60% and about 95% pure.

20. The method according to claim 15, wherein the solvent is selected from the group consisting of C₁-C₈ alcohols, water, acetonitrile, tetrahydrofuran or mixtures thereof.

21. The method according to claim 15, wherein said recrystallization is performed by a process comprising:
i) dissolving or partially dissolving the crude solid in a suitable solvent at an elevated temperature resulting in a solution;
ii) cooling the solution to form highly pure triethylenetetramine diacid salt; and
iii) collecting highly pure triethylenetetramine diacid salt,
wherein the suitable solvent is selected from a polar protic solvent, a polar aprotic solvent, or mixtures thereof.

22. The method according to claim 21, wherein the polar protic solvent is selected from water, methanol, or mixtures thereof.

23. The method according to claim 21, wherein said polar aprotic solvent is acetonitrile or an aqueous solution comprising acetonitrile.

24. The method according to claim 23, wherein said aqueous solution comprising acetonitrile comprises between about 2% and between about 50% water.

25. The method according to claim 22 wherein the solvent is methanol.

26. The method according to claim 21, wherein the elevated temperature is between about ° C. and about the boiling point of the solvent or mixture of solvents.

27. A method for the preparation of a highly pure triethylenetetramine diacid salt comprising the steps of:
a) dissolving impure triethylenetetramine (TET) in a first solvent to form a first solution;
b) warming the first solution to a first temperature;
c) adding a diacid as a solid or in a solution in a second solvent to the first solution at a first temperature to form a second solution;
d) optionally, cooling the second solution to a second temperature to form a precipitate of highly pure triethylenetetramine diacid salt; and
e) collecting the highly pure triethylenetetramine diacid salt.

28. The method according to claim 27, wherein said highly pure triethylenetetramine diacid salt is between about 95% and about 99.95% pure.

29. The method according to claim 27, wherein said diacid is selected from the group consisting of succinic acid, malonic acid, fumaric acid, malic acid, maleic acid, tartaric acid and derivatives thereof, dibenzoyl tartaric acid, oxalic acid, glutaric acid and adipic acid.

30. The method according to claim 29, wherein the diacid is succinic acid.

31. The method according to claim 27 wherein said impure triethylenetetramine (TET) is between about 60% and about 90% pure.

32. The method according to claim 27, wherein said first solvent is selected from the group consisting of methanol, acetonitrile, tetrahydrofuran, water and mixtures thereof.

33. The method according to claim 27, wherein said first temperature is between about 50° C. and about 80° C.

34. The method according to claim 27, wherein said second solvent is selected from the group consisting of methanol, acetonitrile, tetrahydrofuran, water and mixtures thereof.

35. The method according to claim 27, wherein said second temperature is between about −5° C. and about 25° C.

36. A method for the preparation of a highly pure triethylenetetramine dihydrochloride salt comprising the steps of:
a) contacting impure triethylenetetramine (TET) with a diacid to form a crude solid;
b) recrystallizing the crude solid to obtain a highly pure triethylenetetramine diacid salt;
c) contacting the highly pure triethylenetetramine diacid salt with aqueous base to form highly pure triethylenetetramine (TET); and
d) converting the highly pure triethylenetetramine (TET) into its dihydrochloride salt.

37. The method according to claim 36, wherein said highly pure triethylenetetramine diacid salt is between about 95% and about 99.95% pure.

38. The method according to claim 36 wherein said diacid is selected from the group consisting of succinic acid, malonic acid, maleic acid, fumaric acid, malic acid, tartaric acid and derivatives thereof, dibenzoyl tartaric acid, oxalic acid, glutaric acid and adipic acid.

39. The method according to claim 36 wherein the diacid is succinic acid.

40. The method according to claim 36, wherein the aqueous base is selected from the group consisting of aqueous sodium hydroxide, aqueous potassium hydroxide, aqueous sodium carbonate and aqueous potassium carbonate.

41. The method according to claim 36, wherein said recrystallization is performed by the following steps:
i) dissolving or partially dissolving the crude solid in a suitable solvent at an elevated temperature resulting in a solution or suspension;
ii) cooling the solution or suspension to form highly pure triethylenetetramine diacid salt; and
iii) collecting highly pure triethylenetetramine diacid salt.

42. The method according to claim 41, wherein said suitable solvent is selected from the group consisting of polar protic and polar aprotic solvents or mixtures thereof.

43. The method according to claim 41, wherein said polar protic solvent is selected from water, methanol, or mixtures thereof.

44. The method according to claim 41, wherein said polar aprotic solvent is selected from the group consisting of C₁-C₈ ethers, cyclic C₄-C₆ ethers, acetone, 2-butanone, dimethylacetamide, N-methylpyrrolidinone, N, N-dimethylformamide, dimethyl sulfoxide, acetonitrile dimethylpropylene urea, pyridine, sulfolane and hexamethylphosphoric triamide.

45. The method according to claim 44, wherein said polar aprotic solvent is acetonitrile or an aqueous solution comprising acetonitrile,
wherein the aqueous solution comprising acetonitrile comprises between about 2% and between about 25% water.

46. The method according to claim 41, wherein the elevated temperature is between about ° C. and about the boiling point of the solvent or mixture of solvents.

47. A method for the preparation of a highly pure triethylenetetramine dihydrochloride salt comprising the steps of:
a) contacting impure triethylenetetramine (TET) with a diacid to form a crude solid;
b) optionally recrystallizing the crude solid to obtain a highly pure triethylenetetramine diacid salt;
c) contacting the highly pure triethylenetetramine diacid salt with an ion exchange solid to form highly pure triethylenetetramine (TET); and
d) converting the highly pure triethylenetetramine (TET) into its dihydrochloride salt.
